(19) United States
(12) Patent Application Publication
(10) Pub. No.: US 2023/0061069 A1
(43) Pub. Date: Mar. 2, 2023

(54) A PROCESS FOR PRODUCING A PLURALITY OF SUMOYLATION TARGET-SITE MODIFIED AAV VECTOR AND PRODUCT THEREOF

(71) Applicants: Jayandharan Giridhara RAO, Kampur (IN); Shubham MAURYA, Kampur (IN); INDIAN INSTITUTE OF TECHNOLOGY KANPUR, Kanpur (IN)

(72) Inventors: Jayandharan Giridhara Rao, Kanpur (IN); Shubham Maurya, Kanpur (IN)

(21) Appl. No.: 17/293,164

(22) PCT Filed: Oct. 10, 2019
(86) PCT No.: PCT/IB2019/058630

§ 371 Date: May 12, 2021

(30) Foreign Application Priority Data
Nov. 13, 2018 (IN) 201841042550

(51) Int. Cl. C12N 15/86; C07K 14/005

(52) U.S. Cl. CPC C12N 15/86; C07K 14/005; C12N 2750/14122; C12N 2750/14143; C12N 2750/14151; C12N 2750/14171

(57) ABSTRACT

The present invention provides for a process for producing a plurality of SUMOylation target-site modified AAV vectors, SUMOylation target-site modified AAV vectors, and application of the SUMOylation target-site modified AAV vectors in gene therapy. The present invention provides for manipulation of SUMOylation specific amino acids on AAV2 capsid protein, thereby regulating role of the same. Furthermore, the development of SUMOylation target modified AAV2 vectors presents an exciting opportunity for hepatic or ocular gene transfer with the safest AAV vector in human gene therapy applications. The plurality of SUMOylation target-site modified AAV vectors are not immunogenic in comparison to wildtype AAV2 vectors and possess significantly higher gene expression, with respect to wild type, thereby improving efficiency of hepatic and ocular gene transfer.

### FIELD OF THE INVENTION

The present invention is related to the manipulating transduction ability of Adeno-associated virus (AAV), more particularly it relates to a process for producing a plurality of SUMOylation target-site modified AAV vectors and a product thereof.

### BACKGROUND

Gene therapy has become a reliable tool for therapeutic interventions for the phenotypic correction of gene disorders. Several gene therapy systems based on viral and non-viral vectors have been utilized for this purpose [Naldini L. Gene therapy returns to center stage. Nature 2015; 526(7573):351-60]. Among different viral vector-based systems, recombinant Adeno associated viruses (AAV) are appealing due to their better transduction profile across different tissues and their relatively nonpathogenic nature [McCown T. Adeno-Associated Virus (AAV) Vectors in the CNS. Curr Gene Ther 2005; 5(3):333-38]. AAV belongs to the Parvoviridae family and the genus Dependovirus. AAV is a non-enveloped virus of ˜20 nm in size, and the capsid structure has an icosahedral symmetry which contains three major capsid proteins named VP1, VP2, VP3 in a ratio of 1:1:10 along with an assembly activating protein (AAP). AAV has the linear single-stranded genome of ˜4.7 kb in size which is flanked by inverted terminal repeats (ITR). Up to 13 different AAV serotyped have been identified which can transduce a variety of cell types based on their host cellular receptor interaction [Srivastava A. In vivo tissue-tropism of adeno-associated viral vectors. Curr Opin Virol Elsevier B.V. 2016; 21:75-80]. Of these AAV2 is the prototype vector and constitutes a major fraction of AAV serotypes used in human trials thus far. In order to transduce a cell, AAV interacts with cell surface receptors for its endocytosis. Various cell surface receptors and co-receptors have been identified for multiple AAV serotypes.

A recent study also suggested the involvement of a universal receptor for all the serotypes [Pillay S, Meyer N L, Puschnik A S, et al. An essential receptor for adeno-associated virus infection. Nature 2016; 530(7588):108-12]. Upon its entry into the host cell, it traffics towards the nucleus via endosome compartments, and in the process undergoes maturation from early (Rab5+) to late (Rab7+) endosomes [Harbison C E et al., Early Steps in Cell Infection by Parvoviruses: Host-Specific Differences in Cell Receptor Binding but Similar Endosomal Trafficking, J Virol 2009, and Ding W, et al. rAAV2, The American Society of Gene Therapy 2006; 13(4):671-82].

Endosome encapsulated AAV2 particles have been shown to traffic towards Golgi apparatus by various studies. The importance of Golgi apparatus in AAV transduction has also been shown by using small molecule disruptors such as brefeldin A and golgicide A [Douar A et al., Intracellular Trafficking of Adeno-Associated Virus Vectors: Routing to the Late Endosomal Compartment and Proteasome Degradation Intracellular Trafficking of Adeno-Associated Virus Vectors; Routing to the Late Endosomal Compartment and Proteasomc Dcgra. J Virol 2001; 75(4):1824-33; and Nonnenmacher M E et al. Syntaxin 5-dependent retrograde transport to the trans-Golgi network is required for adeno-associated virus transduction. J Virol 2015; 89(3):1673-87].

The fate of AAV2 transduction is also decided by acidification of endosomes as it matures, which alters the VP1 capsid structure to activate its phospholipase A2 (PLA2) activity. Activation of PLA2 facilitates AAV2 to escape endosome compartment in the absence of which endosomes would fuse with lysosome compartments and can cause degradation of AAV2 [Stahnke S. et al., Intrinsic phospholipase A2 activity of adeno-associated virus is involved in endosomal escape of incoming particles, Virology Elsevier Inc. 2011; 409(1):77-83]. These findings are supported by the deletional and mutational studies in the PLA2 domain of the VP1 unique region of AAV2[Girod A. et al., J Gen Virol 2002; 83(5), Stahnke S. et al., Virology Elsevier Inc. 2011; 409(1):77-83; and Grieger J C et al., J Virol 2007; 81(15):7833-43].

A recent study demonstrated that inhibition of endoplasmic reticulum-associated degradation (ERAD) by eeyar-estatin I (EerI) in HeLa cells increases the AAV2 transduction possibly suggesting the role of endoplasmic reticulum in AAV transduction, which was further supported by pharmacological inhibition strategies [Nonnenmacher M E, et al. J Virol 2015; 89(3); Berry G E, et al., J Biol Chem 2015, 291(2)]. After escaping endosome compartment, AAV2 must enter into the nucleus for successful transduction of cell. Post endosomal escape AAV has to enter the nucleus in order to deliver its transgene and expression. Tubulin and actin filaments have been shown to be an important factor for AAV2 nuclear entry.

Furthermore, real-time imaging of fluorescently labeled AAV2 suggested the movement of AAV in the cytoplasm and nucleus can be facilitated by ATP-dependent molecular motors on tubular networks. Multiple studies have suggested that AAV2 might enter the nucleus by alternative pathways as well. Another study has identified the presence of non-conventional nuclear localization signal sequence present in VP2 capsid protein. Post-nuclear entry, AAV2 moves from the nucleolus to nucleoplasm, where it uncoats and initiates gene expression. Given the diversity and potential of cellular proteins known to interact with AAV2, each step in the infection process can be a potential barrier for its successful transduction [Bartlett J S, et al., Infectious entry pathway of adeno-associated virus and adeno-associated virus vectors, J Virol 2000; 74(6):2777-85]. During intracellular trafficking, post-translational modifications (PTMs) like phosphorylation and ubiquitination have been reported to be responsible for viral capsid degradation by initiating proteasome degradation pathways.

To address this rate-limiting step, AAV2 vectors modified at capsid residue determined to be involved in phosphorylation and ubiquitination showed an increase in transduction efficiency [Zhong L, Li B, Mah C S, et al. Next generation of adeno-associated virus 2 vectors: Proc Natl Acad Sci 2008; 105(31) and Gabriel N, Hareendran S, Sen D, et al., Hum Gene Ther Methods 2013; 24(2)]. However, the presence and potential of other PTMs on AAV2 capsid and their possible implication on AAV2 transduction is largely unexplored.

A recent study demonstrated the role of SUMOylation, an Ubiquitin-like modifier (UBL) in the context of AAV2 transduction [Hölscher C et al., The SUMOylation Pathway Restricts Gene Transduction by Adeno-Associated Viruses. PLOS Pathog 2015; 1-23]. Hölscher employed siRNA mediated knockdown of SAE2 and UBC9 target in the SUMOylation pathway and demonstrated an increase in the transduction ability of AAV2 in an in-vitro model. SUMO (small ubiquitin-like modifier) is an 11 kDa protein which shares structural similarity to ubiquitin protein. The process of SUMOylation involves a enzymatic-cascade and involves maturation, activation, conjugation and ligation of SUMO proteins to its substrates. The SUMO proteins mature by C-terminal cleavage mediated by a family of SENP (sentrin/SUMO-specific protease) enzymes. It undergoes an ATP-dependent activation by E1 (SAE1/SEA2) activating enzyme followed by binding to E2 conjugating enzyme (UBC9) via thioester linkage. Finally, it binds to its substrate at consensus ψKXE (where ψ represents a hydrophobic amino acid, and X represents any amino acid) lysine residue with the help of E3 ligases. The SUMOylation is a reversible process which is majorly governed by SUMO proteases. The SUMOylation is a critical process required for normal cell physiology as suggested by knockout studies in DT40 chicken lymphocyte cell line which resulted in abnormalities in chromosomal segregation, nuclear localization and cell death by apoptosis [Hayashi T, et al. Exp Cell Res 2002; 280:212-21].

However, the present state of the art does not provide for the AAV vectors that may demonstrate desirable transduction efficiency both in vitro and in vivo, and where the said AAV vector is also effective in gene therapy.

Therefore, it is required to have AAV vectors which shows improved gene transfer efficiency both in vitro and in vivo during gene therapy.

### SUMMARY OF THE INVENTION

This summary is provided to introduce a selection of concepts, in a simple manner, which are further elaborated in detailed description of the invention. This summary is neither intended to identify the key or essential inventive concept of the subject matter, nor to determine the scope of the invention.

In an embodiment of the present invention, a process for producing a plurality of SUMOylation target-site modified AAV vectors is disclosed. The process includes predicting a plurality of SUMOylation target-sites on AAV VP1 capsid protein sequence based on direct amino acid match to SUMO-consensus sequence and substitution of the consensus amino acid residues with amino acid residues exhibiting similar hydrophobicity. The AAV VP1 capsid protein sequence being set forth in Protein ID-YP_680426.1. The process also includes scoring each of the plurality of SUMOylation target-sites on AAV VP1 capsid protein sequence. The process further includes producing the plurality of SUMOylation target-site modified AAV vectors based on predicted plurality of SUMOylation target-sites on AAV VP1 capsid protein sequence. The production of the plurality of SUMOylation target-site modified AAV vectors comprises following selecting the predicted plurality of SUMOylation target-sites on AAV VP1 capsid protein sequence, having score >0.5, for site-directed mutagenesis, and mutating the selected plurality of SUMOylation target-sites from Lysine (K) to Glutamine (Q) residues to produce plurality of SUMOylation target-site modified AAV vectors.

In another embodiment of the present invention, a plurality of SUMOylation target-sites modified AAV vectors are disclosed, where the AAV vectors are produced by the aforementioned process. The plurality of SUMOylation target-site modified AAV vectors are not immunogenic in comparison to wildtype AAV2 vectors. The said vectors possess significantly higher gene expression, with respect to wild type, thereby improving efficiency of hepatic and ocular gene transfer.

To further clarify the advantages and features of the present invention, a more particular description of the invention will follow with reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE ACCOMPANYING DRAWINGS

The disclosure will be described and explained with additional specificity and detail with the accompanying figures in which:

FIGS. 1A-D illustrate graphs of Quantitative PCR analysis of SUMOylation pathway genes in response to AAV2 infection with respect to SUMO-1, SAE-1, SAE-2, and UBC9, respectively, according to an embodiment of the present invention;

The FIG. 2 is graph plot depicting transduction efficiency of AAV2 mutant vectors in ARPE 19 cells in vitro, in the context of the invention;

FIGS. 3A and B are graph plots illustrating Transduction efficiency of AAV2 mutant vectors in Huh7 and HeLa cells in vitro, respectively, in the context of the invention;

FIG. 4 is a graph depicting entry profile of AAV2 wild-type and K105Q mutant vector in HeLa cells in vitro, in the context of the present invention;

FIG. 5 is graph plot showing a virus neutralization assay, in the context of the invention.

FIG. 6 is western blot analysis for wild type and mutant vector for capsid SUMOylation, in the context of the invention;

FIG. 7 is graph illustration for efficiency of SUMOylation site modified AAV2 vectors for gene transfer in a murine model of hemophilia B, in the context of the invention;

FIG. 8 is micrographs for immunohistochemistry for human factor IX after hepatic gene transfer in vivo, in the context of the invention;

FIG. 9 is graph illustration for immune response in hemophilia B mice administered with AAV2 wild type or mutant vectors, in the context of the invention;

FIG. 10 is graph illustration IFN-γ based ELISPOT assay to measure AAV2 capsid specific T-cell response, in the context of the invention;

FIGS. 11A-B are images of Ocular gene transfer in C57BL6/J mice with SUMOylation mutant vector, respectively for 2 and 8 weeks, in the context of the invention;

FIG. 11C is graph plot between mean GFP intensity and time plot analysis for Ocular gene transfer in C57BL6/J mice with SUMOylation mutant vector, respectively for 2 and 8 weeks, in the context of the invention;

FIG. 12A are images depicting Ocular gene transfer in C57BL6/J mice with SUMOylation mutant vector four weeks after gene transfer, in the context of the invention;

FIG. 12B is graph plot illustrating Ocular gene transfer in C57BL6/J mice with SUMOylation mutant vector four weeks after gene transfer, in the context of the invention; and

FIG. 13 is a graph plot illustrating electroretinograms after ocular gene transfer in rd12 mice with SUMOylation mutant vector ten weeks after gene transfer, in the context of the invention.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE INVENTION

For the purpose of promoting an understanding of the principles of the invention, reference will now be made to the embodiments illustrated in the figures and specific language will be used to describe them. It will nevertheless be understood that no limitation of the scope of the invention is thereby intended. Such alterations and further modifications in the illustrated process, and such further applications of the principles of the invention as would normally occur to those skilled in the art are to be construed as being within the scope of the present invention.

It will be understood by those skilled in the art that the foregoing general description and the following detailed disclosure are exemplary and explanatory of the invention and are not intended to be restrictive thereof.

The terms “comprise”, “comprising”, or any other variations thereof, are intended to cover a non-exclusive inclusion, such that a process or method that comprises a list of steps does not include only those steps but may include other steps not explicitly listed or inherent to such a process or method. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this invention belongs. The system, methods, and examples provided herein are only illustrative and not intended to be limiting.

Embodiments of the present invention will be described below in detail with reference to the accompanying figures.

The present invention provides for a process for producing a plurality of SUMOylation target-site modified AAV vectors and SUMOylation target-site modified AAV vectors. The present invention also talks about application of the SUMOylation target-site modified AAV vectors in ocular and hepatic gene therapy.

Adeno-associated virus (AAV) based gene delivery vectors have become a predominant tool for multiple gene therapy applications over the years. However, barriers related to reduced transduction with recombinant AAV2 vectors and the host immune response directed against the AAV2 capsid proteins are significant limitations that prevent its extensive use in humans. Molecular signatures such as various post-translational modifications (PTM) imprinted onto AAV2 vectors during its packaging or its infection process are known to impact the transduction ability of these vectors. Among the plethora of PTMs, SUMOylation has been discovered as an inhibitory pathway for AAV2 transduction (Hölscher et al, 2015) and is also known to manipulate the cellular immune response against other viruses such as human papillomavirus (HPV), Dengue virus (DENV) and Influenza A virus. The present invention provides for modification of SUMOylation sites on AAV2 which may circumvent SUMOylation of its capsid protein and improve AAV2 transduction. During experiments conducted in the context of the present invention a targeted screen of SUMOylation genes in AAV2 infected HeLa cells was performed. Data collected from the experiments showed that SUMOylation pathway genes (SUMO Activating Enzyme Subunit 1 (SAE1), SUMO Activating Enzyme Subunit 2 (SAE2), UBC9, and Small Ubiquitin-Like Modifier 1 (SUMO-1)) are significantly up-regulated (2 to 6-fold) within 6 hours' time-point of AAV2 transduction.

In an embodiment of the present invention, a process for producing a plurality of SUMOylation target-site modified AAV vectors is disclosed. The process includes predicting a plurality of SUMOylation target-sites on AAV VP1 capsid protein sequence based on direct amino acid match to SUMO-consensus sequence and substitution of the consensus amino acid residues with amino acid residues exhibiting similar hydrophobicity. The AAV VP1 capsid protein sequence being set forth in Protein ID-YP_680426.1. The process also includes scoring each of the plurality of SUMOylation target-sites on AAV VP1 capsid protein sequence. The process further includes producing the plurality of SUMOylation target-site modified AAV vectors based on predicted plurality of SUMOylation target-sites on AAV VP1 capsid protein sequence. The production of the plurality of SUMOylation target-site modified AAV vectors comprises following selecting the predicted plurality of SUMOylation target-sites on AAV VP1 capsid protein sequence, having score >0.5, for site-directed mutagenesis, and mutating the selected plurality of SUMOylation target-sites from Lysine (K) to Glutamine (Q) residues to produce plurality of SUMOylation target-site modified AAV vectors.

In an embodiment of the present invention, the AAV used herein is selected from a group consisting AAV serotypes 1, 2, 3, 4, 5, 6, 7, 8, 9, rh10 and other AAV variants thereof.

In a specific embodiment, the AAV used herein is AAV2.

The present invention provides for identifying, based on in silico tools, the SUMOylation sites on AAV2 capsid protein. Totally 13 sites were identified, based on the identified sites top 5 [K26, K39, K105, K527, K620] mutant capsids were generated by site directed mutagenesis of the AAV2 vector and tested the mutant AAV2 vectors K26, K39, K105, K527, and K620 in vitro. It was further identified that one these mutant AAV2 vectors, K105Q showed a significant increase in transduction in multiple cell lines, i.e., HeLa (up to 97%), Huh7 (up to 44%), and ARPE19 (up to 110%).

The mutant AAV2 vectors were further evaluated for gene therapy, in vivo, in hemophilia B mice. It was identified that the AAV2-K105Q mutant vector expressing human coagulation factor (F) IX had a 1.72 to 2-fold increase in FIX levels at 5 to 8 weeks after hepatic gene therapy. To further test its potential in ocular gene therapy, intravitreal administration of AAV2-K105Q mutant vectors expressing GFP in comparison to wildtype AAV2 was performed. A modest increase (up to 2.15-fold) in ocular transduction with AAV2-K105Q vectors in C57BL6 mice was observed. To further evaluate the therapeutic efficiency of AAV2-K105Q vectors for ocular gene therapy, AAV2 wild type and K105Q expressing human retinal pigmental epithelium gene encoding 65 kDa protein (RPE65) were administered in groups of rd12 mice. Approximately, 1-2 μl of vectors containing 7×10⁸ vgs were administered by subretinal route into the eyes of rd12 mice. The phenotypic rescue was measured by electroretinography (ERG) analysis 10 weeks after vector administration. The A-wave amplitude for K105Q vector administered mice was significantly elevated to −63±9.3 μV in comparison to eyes that received AAV2 wild type vectors (−25.07±12.69 μV) and mock injected animals (−13.4±2.0 μV). The B-wave amplitude for AAV2 K105Q administered and AAV2 wild type administered eyes were 93.69±2.0 μV and 55.07±15.12 μV, respectively. This highlights that ocular gene therapy with AAV2 K105Q-RPE65 vectors has a therapeutic A-wave amplitude response and B-wave response when compared to rd12 mice that received wild type AAV2 vectors. Therefore, the present invention demonstrates the therapeutic potential of SUMOylation site modified AAV2 vectors for ocular or hepatic gene therapy applications.

The present invention provides for identification that many SUMOylation genes (Table-1) are inhibitory to AAV2 transduction process. Based on this preliminary analysis, we have generated novel AAV2 vectors that demonstrate improved transduction efficiency both in vitro and in vivo.

In an embodiment, the present invention provides for identification that many SUMOylation genes (Table-1) are inhibitory to AAV2 transduction process based on secondary analysis of publicly available RNAi screening data from Miguel Mano et al. 2015. Genes involved in SUMOylation pathway were retrieved from Reactome pathway database (R-HSA-2990846) and were matched against the host factors identified in AAV2 infection through RNAi analysis. For the screening, the authors utilized siRNA library against 18,120 human genes in HeLa cells which was then transduced with single stranded AAV2-Luciferase vectors.

Further, the transduction efficiency was determined in presence of each of the specific siRNA/target gene. A total of 1,483 cellular factors were proposed to be significantly affecting, either positively or negatively, the AAV2 vector transduction efficiency. The present invention generated a plurality of novel AAV2 vectors that demonstrate improved transduction efficiency both in vitro and in vivo, based on the present analysis.

TABLE 1

| | Role in | |
|---|---|---|
| Gene | AAV2 | |
| ID | transduction | Description |
| RAE1 | Negative | ribonucleic acid export 1 |
| PIAS4 | Negative | protein inhibitor of activated STAT 4 |
| ZNF131 | Negative | zinc finger protein 131 |
| PCSK2 | Negative | proprotein convertase subtilisin/kexin type 2 |
| NUP37 | Negative | nucleoporin 37 |
| NR1H3 | Negative | Nuclear Receptor Subfamily 1 Group H |
| | | Member |
| NRIP1 | Negative | nuclear receptor interacting protein 1 |
| UBE21 | Negative | ubiquitin conjugating enzyme E2 1 |
| UBA2 | Negative | ubiquitin like modifier activating enzyme 2 |
| MDC1 | Negative | mediator of DNA damage checkpoint 1 |
| PCNA | Negative | proliferating cell nuclear antigen |
| SAE1 | Negative | SUMO1 activating enzyme subunit 1 |
| HDAC1 | Negative | histone deacetylase 1 |
| PHC3 | Negative | polyhomeotic homolog 3 |
| CASP8AP2 | Negative | Caspase 8 Associated Protein 2 |
| HIC1 | Positive | HIC ZBTB transcriptional repressor 1 |
| DNMT3B | Positive | DNA methyltransferase 3 beta |
| NUP88 | Positive | nucleoporin 88 |
| CETN2 | Positive | centrin 2 |

### Cell Lines and Animal Models

Huh-7 cell line used in the context of the present invention was received from Dr. Saumitra Das, IISc, Bangalore. HeLa cell line was obtained from American Type Culture Collection (ATCC, Manassas, USA). ARPE19 cell line was received from Dr. Sowmya Parameswaran and Dr. Krishnakumar, Sankara Nethralaya, Chennai. The cells were cultured in complete Tscove's Modified Dulbecco's Medium (IMDM) (Gibco, Life Technologies, Carlsbad, USA) with 10% Fetal Bovine Serum (Gibco, Life Technologies, Carlsbad, USA) at 37° C. with 5% CO₂, supplemented with Ciprofloxacin (HiMedia Laboratories, Mumbai, India) and Piperacillin (MP Biomedicals, LLC, USA) at 10 μg/ml each. Intravenous immunoglobulins (IVIG) was procured from Baxter Biosciences (Deerfield, USA). SYBR green qPCR mastermix was purchased from Promega (Madison, USA). C57BL6/J and Factor-IX deficient mice (B6.129P2-F9^{tm1Dws}/J) was procured from Jackson Laboratory (Bar Harbor, USA). All animal experiments were approved by the IIT-Kanpur Institutional Animal Ethics committee.

In one embodiment, the present invention provides for the method for developing the SUMOylation target-site modified AAV vectors. The method includes targeted transcriptome analysis for SUMOylation pathway, in silico prediction for SUMOylation sites on AAV2 VP1 capsid protein, and vector production based on the same.

### Targeted Transcriptome Analysis for SUMOylation Pathway

In one embodiment, total RNA from each of the treated condition in HcLa cells was extracted by TRIzol reagent (Thermo Fisher, Waltham, USA). About 1 μg of RNA was used to generate cDNA by using the Verso cDNA synthesis kit (Thermo Fisher). The primers used herein for SUMOylation gene targets were procured from Imperial Life Sciences (ILS, Gurgaon, India). Transcript levels of SUMOylation target genes SUMO Activating Enzyme Subunit 1 (SAE1), SUMO Activating Enzyme Subunit 2 (SAE2), UBC9, and SUMO-1 (Table-2) were measured by quantitative (q)PCR in a CFX97 Real-Time system (Biorad, Hercules, USA) with beta-actin as an endogenous control for the normalization of output data.

TABLE 2
Primers designed for analysing gene expression of SUMOylation pathway genes.

| Enzyme | | | | | |
|---|---|---|---|---|---|
| targets in | | | | | |
| SUMOylation | | | Length | Tm | Amplicon |
| pathway | Name | Primer | (bp) | (° C.) | length(bp) |
| E1 | SAE1 | 5′-AGTCATTTTTCACTCAATTCGATGCT-3′ | 26 | 58.3 | 122 |
| | | 5′-GCCAAAAACATCTCCTGTAAAGAAC-3′ | 25 | 57.1 | |
| E1 | SAE2 | 5′-GCTGGGTATCTTGGACAAGTAACT-3′ | 24 | 58.1 | 92 |
| | | 5′-CCAGGAAAGGTTCTCTGGGTC-3′ | 21 | 57.1 | |
| E2 | UBC9 | 5′-GCCATTCCAGGAAAGAAAGGGA-3′ | 22 | 57.9 | 90 |
| | (UBE2I) | | | | |
| | | 5′TGGTGGCGAAGATGGATAATCATC-3′ | 24 | 58.6 | |
| | SUMO1 | 5′GGCAAAACCTTCAACTGAGGAC-3′ | 22 | 57.4 | 91 |
| | | 5′-GTGAATCTCACTGCTATCCTGTCC-3′ | 24 | 58.6 | |

### In Silico Prediction for SUMOylation Sites on AAV2 VP1 Capsid Protein

In one embodiment, AAV2 VP1 capsid protein sequence (Protein ID-YP_680426.1) was used to predict SUMOylation targets with the online tools GPS-SUM and SUMOplot (http://www.abgent.com/sumoplot). GPS-SUMO is based on the fourth-generation GPS algorithm integrated with the Particle Swarm Optimization (PSO) method for predicting the SUMOylation sites and SUMO plot is based on two criteria: direct amino acid match to SUMO-consensus sequence and substitution of the consensus amino acid residues with amino acid residues exhibiting similar hydrophobicity.

### Vector Production

The top 5 sites predicted with a score>0.5 as SUMOylation targets in AAV2 capsid by both GPS-SUMO and SUMOplot were chosen for further site-directed mutagenesis (Table-3). SUMOylation targets were mutated from Lysine (K) to Glutamine (Q) residues by using Quick-change II XL site-directed mutagenesis kit (Agilent Technologies, Santa Clara, USA) as per the manufacturer's instructions. Viral vectors were packaged and purified as described by Ling C, et al. J Vis Exp 2011. Briefly, forty numbers of 150-mm² dishes, 80% confluent with AAV-293 cells were transfected with AAV2 (rep/cap) or AAV2 mutant capsid vectors, transgene (p.dsAAV2-EGFP) vectors and AAV-helper (p.helper) vectors. Cells were collected 68-72 hours post-transfection, lysed and treated with benzonase nuclease (25 units/ml; Sigma-Aldrich, St. Louis, USA). Further, the vectors were purified by iodixanol gradient ultracentrifugation (OptiPrep; Sigma-Aldrich) followed by column chromatography (HiTrap SP column; GE Healthcare Life Sciences, Chicago, USA). The vectors were concentrated to a final volume of 0.5 ml in phosphate-buffered saline (PBS), using Amicon Ultra 10K centrifugal filters (Millipore Burlington, USA). Physical particle titers were quantified by qPCR as described by Aurnhammer C., et al. 2012.

TABLE 3
Primers designed for mutagenesis of predicted SUMOylation sites from lysine
to glutamine residue. The bold face and underlined font
indicate the site of mutation.

| | | Seq. | | | |
|---|---|---|---|---|---|
| | | ID | | Tm | GC |
| Mutant | Primer | No. | bp | (° C.) | % |
| K26Q | 5′-TTTGGTGGTGGTGGGCCAGGTTGGAGCTTCCACCACTGT-3′ | 1 | 39 | 88 | 58 |
| | 5′-ACAGTGGTGGAAGCTCCAACCTGGCCCACCACCACCAAA-3′ | 2 | | | |
| K39Q | 5′-CCTGCTGTCGTCCTGATGCCGCTCTGCGGGCTTT-3′ | 3 | 34 | 87 | 64 |
| | 5′-AAAGCCCGCAGAGCGGCATCAGGACGACAGCAGG-3′ | 4 | | | |
| K105Q | 5′-CCCCAAAAGACGTATCTTCTTGAAGGCGCTCCTGAAACTCC-3′ | 5 | 41 | 84 | 53 |
| | 5′-GGAGTTTCAGGAGCGCCTTCAAGAAGATACGTCTTTTGGGG-3 | 6 | | | |
| K527Q | 5′-AACTTTTCTTCATCGTCCTGGTGGCTTGCCATGGCCGGG-3′ | 7 | 39 | 88 | 56 |
| | 5′-CCCGGCCATGGCAAGCCACCAGGACGATGAAGAAAAGTT-3′ | 8 | | | |
| K620Q | 5′-GTCCGTGTGTGGAATCTGTGCCCAGATGGGCCCCTGAAG-3′ | 9 | 39 | 89 | 61 |
| | 5′-CTTCAGGGGCCCATCTGGGCACAGATTCCACACACGGAC-3′ | 10 | | | |

In another embodiment of the present invention, a plurality of SUMOylation target-site modified AAV vectors are disclosed, where the AAV vectors are produced by the aforementioned process. The plurality of SUMOylation target-site modified AAV vectors are not immunogenic in comparison to wildtype AAV2 vectors. The said vectors possess significantly higher gene expression, with respect to wild type, thereby improving efficiency of hepatic and ocular gene transfer.

### Transduction Assays In Vitro

About, 3×10⁴ cells per well were seeded in a 24 well plate and incubated for 12 hours in IMDM with 10% fetal bovine serum in humidified conditions and 5% CO₂. HeLa (Human cervical carcinoma), Huh7 (Human hepatocellular carcinoma) and ARPE19 (Adult Retinal Pigment Epithelial cell line-19) were mock infected or infected with scAAV2-EGFP and scAAV2-EGFP mutant vectors for at a multiplicity of infection of 5×10³ vgs/cell for 3 hours Two days later, the transgene (GFP) expression was quantified by flow-cytometry (BD Accuri C6 plus, Franklin Lakes, USA).

### Virus Entry Assay

HeLa cells were seeded at a density of 1×10⁵ cells per well in a 24 well plate. Cells were then mock infected or infected with scAAV2-EGFP and mutant viruses at a MOI of 1×10⁴ vgs/cell. 3 hours later, infected cells were collected by trypsinization and genomic DNA was isolated by ethanol precipitation. Viral genomes were quantified against appropriate plasmid standards and with the PolyA site as a target for amplification by qPCR.

### Neutralization Assay

Approximately 3×10⁴ HeLa cells were seeded per well in triplicates and incubated for 12 hours in IMDM with 10% fetal bovine serum in humidified conditions and 5% CO₂. For this assay, the AAV2 vectors were used at a MOI of 5×10³ vgs/cell. The scAAV2-EGFP and the mutant vectors were incubated with 1:256 dilution of human intravenous immunoglobulin (WIG) (10 mg/ml) (Baxter, Deerfield, USA) for 1 hour at 37° C. The choice of this specific dilution of IVIG is based on concentration of IVIG which inhibited scAAV2-EGFP transduction by 50%. Subsequently, cells were infected with mutant vectors and scAAV2-EGFP alone or with vectors that were pre-incubated with IVIG. Forty-eight hours later, the transgene (GFP) expression was quantified by flow cytometry (BD accuri).

In another embodiment, the present invention provides for application of the SUMOylation-target modified AAV2 vectors in hepatic gene therapy.

### Hepatic Gene Transfer of Human Coagulation Factor IX in Hemophilia B Mice

About 5×10¹⁰ vector genomes of self-complementary AAV2 vectors containing LP1 promoter driven human coagulation Factor (F) IX (scAAV2-LP1-F-IX) or scAAV2-K105Q-LP1-F-IX vectors were administered into hemophilia B mice (n=5 animals per group) intravenously. PBS was injected into control haemophilia B mice. Five weeks after gene transfer, a retro-orbital blood collection from all animals was performed and plasma isolated by standard methods. To assay the F-IX activity in murine plasma, an Enzyme-linked immunosorbent assay (ELISA) was performed using a commercial kit (Asserachrom IX: Ag, Diagnostica Stago, Asnieres, France). Briefly, 50 μL of control and animal plasma was incubated for 1 hour at room temperature. Further, wells were washed 5 times with wash buffer and incubated with secondary antibody conjugated with HRP for 1 hour. Wells were washed 5 times with wash buffer and absorbance values were determined by calorimetric color development with 3,3′,5,5′-Tetramethylbenzidine (TMB) substrate followed by spectrophotometric reading at 450 nm. Appropriate quality control samples were employed as suggested by the manufacturer's protocol.

In yet another embodiment, the present invention provides for application of the SUMOylation-target modified AAV2 vectors in ocular gene therapy.

### Western Blot Analysis

About 1.42×10¹⁰ vgs. of AAV vectors were loaded onto a denaturing SDS-PAGE gel. Resolved proteins were further transferred into PVDF membrane (Pall Corporation, New York, USA). Subsequently, the membrane was blocked with 5% BSA for 1 hour Membranes were then probed with anti-AAV (B1) (1:500, Fitzgerald, North Acton, Mass., USA) or anti-SUMO-1 (1:1000, Sigma-Aldrich) primary antibodies and detected with an anti-mouse HRP conjugated secondary antibody (1:2500, Abeam, Milton, Cambridge, UK). The signals were developed by chemiluminescent substrate (SuperSignal™ West Pico PLUS, Thermo Scientific). Densitometric quantification was performed by using Image J in three different blots with two measurements at least for each blot developed.

### Immunohistochemistry

For immunostaining of human FIX, murine liver samples were harvested after 9 weeks of hepatic gene transfer. Samples were embedded in OCT media (Polyfreeze, Sigma Aldrich), sectioned at 10 μM thickness and fixed in 4% paraformaldehyde for 15 mins at room temperature. Slides were washed with PBS and blocked in a solution containing 10% normal donkey serum (Santa Cruz Biotechnology, Dallas, Tex., USA), 0.2% Triton X-100 (Sigma Aldrich) diluted in PBS for 1 hour at room temperature. Subsequently, sections were incubated with goat anti-human FIX antibody (1:100, Affinity Biologicals, Hamilton, ON, Canada) overnight at 40 C. After washing thrice, the slides were incubated with donkey anti-goat Cy3 antibody (Jackson ImmunoReasearch, West Grove, Pa., USA,) at dilution of 1:500 for 1.5 hrs. at room temperature. Sections were washed thrice and mounted with Fluoroshield™ with DAPI (Sigma Aldrich). Images were acquired by Leica DMi8 confocal microscope (Wetzlar, Germany).

### Immune Assays

To examine the immunogenicity associated with hepatic gene transfer of AAV2 vectors, we enumerated the T-cell, B-cell and T-reg cells in hemophilia B mice that received h.FIX gene therapy. Briefly, peripheral blood from hemophilia B mice was collected 9 weeks after gene transfer. After RBC lysis (155 mM NH4Cl, 12 mM NaHCO3& 0.1 mM EDTA), samples were incubated with a combination of FITC labelled anti-CD3, PE-labelled anti-CD8, PerCP labelled anti-CD4 and APC labelled anti-CD19 (BD Biosciences) antibodies for 30 mins at room temperature and percentage CD3+, CD4+, CD8+, and CD19+ cells were assessed by flow cytometry (BD Accuri C6 Plus). To profile the T-reg cells in murine splenocytes, —2 million cells were stained with PerCP labelled anti-CD4 and APC labelled anti-CD25 and PE-conjugated Foxp3 antibodies as per the manufacturer's protocol (BD Biosciences).

ELIspot Assay

Splenocytes from control or treated mice were harvested and samples processed as described earlier. Briefly, after RBC lysis, ˜1×10⁶ viable splenocytes were stimulated with 2 μg/mL of AAV2 capsid T-cell epitope specific peptide (SNYNKSVNV) (JPT Peptide Technologies, GmbH, Germany) and seeded into IFN-γ antibody pre-coated ELISPOT plate (MabTech, Ohio, USA). Concanavalin A (2 μg/mL) was used as positive control for the assay. After 36 hrs of incubation at 37° C., spots were developed using BCIP/NBT. Spot forming units (SFU) and the images were captured in an ELISPOT reader (AID reader, GmbH, Germany).

### Ocular Gene Transfer and Fluorescence Imaging

Eyes of C57BL6/J mice were dilated by 1% Atropine (Jawa Pharmaceuticals India Pvt. Ltd, Gurgaon, India), Phenylephrine+Tropicamide (Sunways India Pvt. Ltd. Mumbai, India). Mice were anesthetized by intraperitoneal injection of ketamine (80 mg/kg) and Xylazine (12 mg/kg). For intravitreal administration, an opening was created at scelera near limbus by an insulin syringe, and 1 μl of AAV vector was injected through the same opening by Hamilton syringe fitted with 33 gauge beveled needle. After injections were completed, tobramycin (Sunways India Pvt. Ltd.) was applied to the eyes. Fluorescence imaging was performed after 2, 4 and 8 weeks of vector administration, in Micron IV imaging system as per manufacturer's instructions (Phoenix Research Lab, Pleasanton, USA). Briefly, eyes of C57BL6/J mice were dilated by 1% Atropine (Jawa Pharmaceuticals India Pvt. Ltd), Phenylephrine+Tropicamide (Sunways India Pvt. Ltd.). Mice were anesthetized by intraperitoneal injection of ketamine (80 mg/kg) and xylazine (12 mg/kg). Further, 2.5% hypromellose (OCuSOFT, Rosenberg, USA) was applied to the eye before imaging. Intensity was set at maximum and gain was set at 15 db or 18 db, the frame rate was set at 6 fps or 4 fps for imaging of all the groups. The fluorescence in the eyes of treated animals was further quantified by Image J analysis as described by Schneider C A. et al., 2012 and Wassmer S J., et al. 2017.

### Electroretinography

For scotopic ERG measurement, mice were dark-adapted overnight. ERG was recorded as per the manufacturer's instruction (Phoenix research lab, Pleasanton, Calif., USA). Briefly, mice were anesthetized with an intraperitoneal injection of ketamine (80 mg/kg) and Xylazine (12 mg/kg) followed by pupil dilation by Phenylephrine+Tropicamide (Sunways India Pvt. Ltd. Mumbai, India). Mice were placed on a heating pad and the reference electrode was subcutaneously placed under the forehead between the ears, while the ground electrode was placed under the tail subcutaneously. Corneal electrode was placed on the cornea after applying 2.5% Hypromellose (OCuSOFT, Rosenberg, N.C., USA). ERG was recorded with the intensity of light flash varying between −1.7 to 3.1 log cd sec/m2.

### Statistical Analysis

Statistical analysis was performed by either Student's t-test or ANOVA by GraphPad Prism 7 (GraphPad, La Jolla, USA). Values obtained between the test and control groups were considered to be statistically significant if the p-value was <0.05. p values at various confidence intervals are denoted as *p<0.05, **p<0.01, ***p<0.001.

### Results

### Specific Genes in the SUMOylation Pathway are Dysregulated Upon AAV2 Infection in HeLa Cells

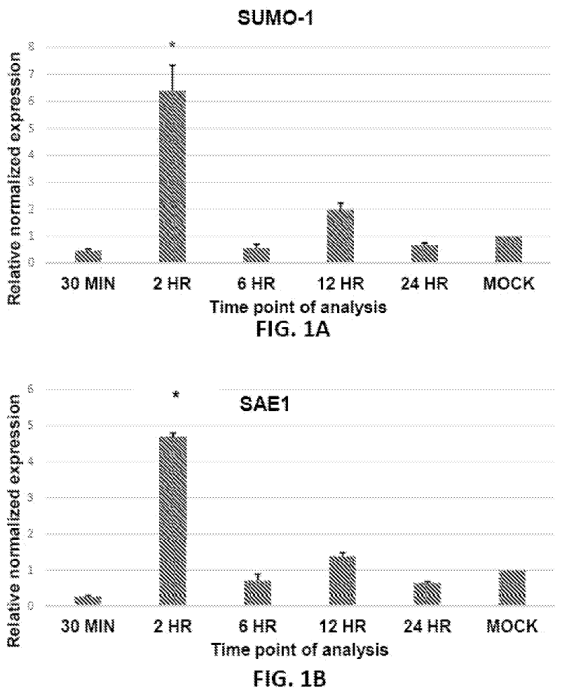

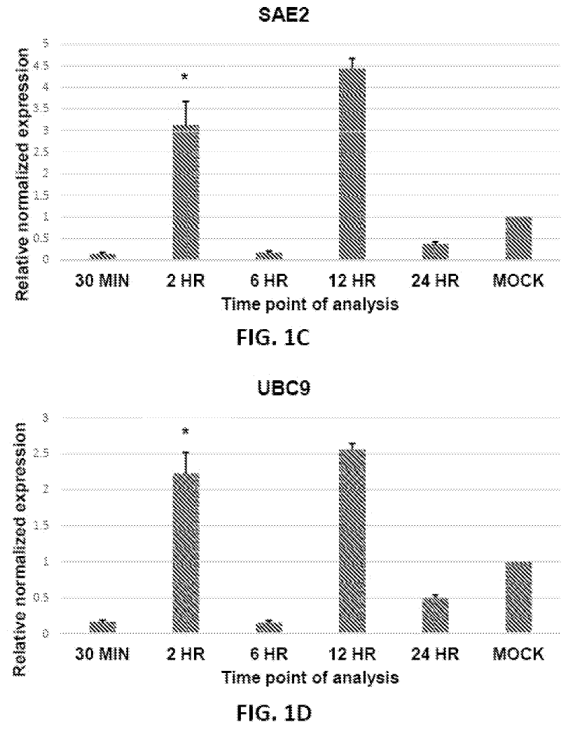

To determine the changes to SUMOylation machinery during AAV2 infection, transcript levels of SUMOylation pathway genes were examined by measuring the expression pattern of marker genes, including activating enzyme (E1), a conjugating enzyme (E2) and the SUMO-1 gene that are known to be important in regulating this pathway. Since multiple ligating enzymes (E3) are known to be important for SUMOylation in a substrate-specific manner, the mRNA level for E3 enzyme was not assessed. A time course analysis of the target genes in HeLa cells at 30 minutes, 2 hours, 6 hours, 12 hours, and 24 hours after AAV2 infection was performed. The data generated by experiments in the context of the invention shows that most of the SUMOylation genes such as SAE1 (4.69-fold), SAE2 (3.12-fold), UBC9 (2.22-fold) SUMO-1 (6.36-fold) are upregulated as early as the 2 hours' time point after infection (FIGS. 1A-D). Several studies [Bartlett J S. et al., 2000 and Xiao P J. et al., 2012], have indicated that AAV2 undergoes cytoplasmic trafficking during this time point in HeLa cells and thus it is conceivable that the changes in transcript levels of SUMOylation genes indicate that this signalling pathway is activated upon AAV2 infection.

FIGS. 1A-D illustrate graphs of Quantitative PCR analysis of SUMOylation pathway genes in response to AAV2 infection with respect to SUMO-1, SAE-1, SAE-2, and UBC9, respectively, according to an embodiment of the present invention. Approximately 8×10⁴ HeLa cells were seeded in 12 well plate and incubated for 12 hours in Iscove's-modified Dulbecco's medium (IMDM) with 10% fetal bovine serum in humidified conditions and 5% CO₂. Cells were either mock-treated or treated with scAAV2-EGFP at a MOI of 5×10³ vgs/cell. Total RNA from each of the treated condition in HeLa cells, was extracted by TRIzol. About 1 μg of RNA was used to convert cDNA. Transcript levels of SUMOylation target genes SAE1, SAE2, UBC9, SUMO-1 (Table 2) were measured by quantitative (q)PCR in a CFX97 Real Time with beta-actin as an endogenous control for the normalization of data. Each time point of analysis had its own mock control. Data presented in FIGS. 1A-D is a mean of 3 replicates. Error bars represent SD, n=3, *P≤0.05

TABLE 4a
SUMOylation sites predicted by GPS-SUMO on
AAV2 VP1 capsid protein. *The numbering of amino
acids in VP1 capsid protein is according to
NCBI Protein ID- YP 680426.1. Indicated region
denotes consensus motif for SUMOylation.

| S.No. | Position* | Peptide | Score |
|---|---|---|---|
| 1 | 105 | 98-AEFQERLKEDTSFGG-112 | 37.13 |
| 2 | 137 | 130-GLVEEPVKTAPGKKR-144 | 38.488 |
| 3 | 527 | 520-GPAMASHKDDEEKFF-534 | 42.941 |

### AAV2 Mutant Vectors Modified at SUMOylation Sites Demonstrate an Increase in Transduction Efficiency In Vitro

The top five residues identified by both GPS-SUMO and SUMOplot (Table-4a, Table-4b) for further mutagenesis in AAV2 capsid were shortlisted. These included lysine residues at K26, K39, K105, K527, K620 in the VP1 protein, that were mutated to corresponding glutamine residues.

TABLE 4b
SUMOylation sites predicted by SUMOplot on AAV2 VP1
capsid protein. *The numbering of amino acids in
VP1 capsid protein is according to NCBI Protein
ID- YP_680426.1. Indicated region denotes
consensus motif for SUMOylation.

| S.No. | Position* | Peptide | Score |
|---|---|---|---|
| 1 | 105 | 99-EFQER LKED TSFGG-112 | 0.91 |
| 2 | 640 | 634-MGGFG LKHP PPQIL-647 | 0.8 |
| 3 | 26 | 20-RQWWK LKPG PPPPK-33 | 0.73 |
| 4 | 620 | 614-QGPIW AKIP HTDGH-627 | 0.69 |
| 5 | 527 | 52-PAMAS HKDD EEKFF-534 | 0.52 |
| 6 | 39 | 33-KPAER HKDD SRGLV-46 | 0.52 |
| 7 | 544 | 538-GVLIF GKQG SEKTN-551 | 0.5 |
| 8 | 314 | 308-PKRLN FKLF NIQVK-321 | 0.5 |
| 9 | 161 | 155-SSSGT GKAG QQPAR-168 | 0.5 |
| 10 | 61 | 55-PFNGL DKGE PVNEA-68 | 0.5 |
| 11 | 143 | 137-KTAPG KKRP VEHSP-150 | 0.37 |
| 12 | 532 | 526-HKDDE EKFF PQSGV-539 | 0.15 |

The average viral titers for these mutants were not significantly different front wild type-vectors (Table-5). These vectors were further tested at a MOI of 5000 in retinal (ARPE19) cells.

TABLE 5
Physical particle titers for the vectors generated
in this study. The data is presented in vector
genomes per ml (vgs/ml).

| | |
|---|---|
| scAAV2-EGFP | 2.00E+11 |
| scAAV2 K26Q-EGFP | 8.71E+11 |
| scAAV2 K39Q-EGFP | 5.45E+11 |
| scAAV2 K10Q-EGFP | 4.69E+11 |
| scAAV2 K527Q-EGFP | 1.38E+12 |
| scAAV2 K620Q-EGFP | 4.14E+11 |

The data from flow cytometry analysis showed a significantly higher (50.1%±11.6% vs. 23.81%±1.1%) increase in EGFP gene expression in scAAV2-K105Q mutant vector-treated cells in comparison to WT-AAV2 vector infected ARPE19 cells (FIG. 2).

The FIG. 2 is graph plot depicting transduction efficiency of AAV2 mutant vectors in ARPE 19 cells in vitro, in the context of the invention. About, 3×10⁴ ARPE19 cells per well were seeded in 24 well plate and incubated for 12 hours in IMDM with 10% fetal bovine serum. Cells were mock infected or infected with scAAV2-EGFP and scAAV2-EGFP mutant vectors for 3 hours Forty-eight hours later, the GFP expression was quantified by flow cytometry. Data presented in the FIG. 2 is a mean of 3 replicates. Error bars represent SD, n−3, ***P≤0.001.

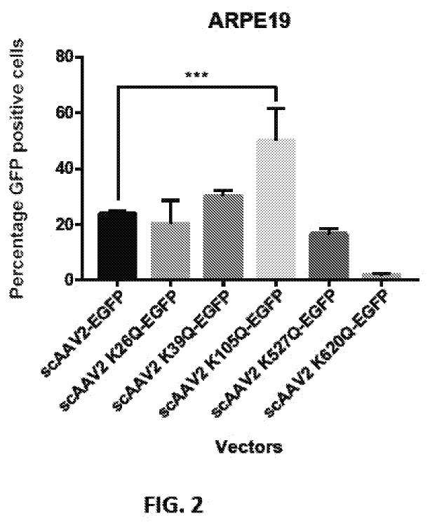

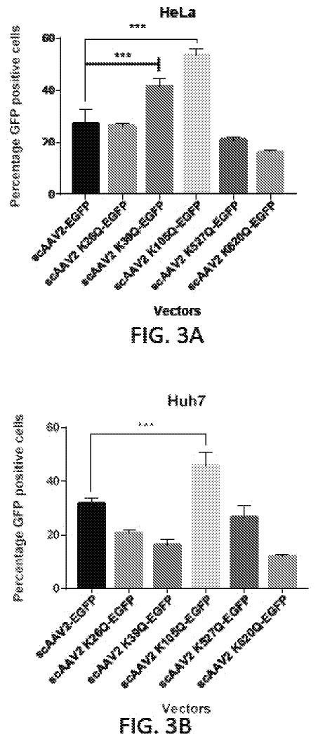

As shown in FIGS. 3A-B, the K105Q mutant demonstrated a similar increase in transgene expression in either HeLa (53.4%±2.4% vs. 27.01%±5.5%) or Huh7 cells (45.7%±5.3% vs. 31.64%±2.0%) suggesting that the mutant vector had higher infectivity consistently in all these cell types. FIGS. 3A and B are graph plots illustrating Transduction efficiency of AAV2 mutant vectors in Huh7 and HeLa cells in vitro, respectively. About, 3×10⁴ HeLa and Huh7 cells per well were seeded in 24 well plate and incubated for 12 hours in IMDM with 10% fetal bovine serum. Cells were mock infected or infected with scAAV2-EGFP and scAAV2-EGFP mutant vectors for 3 hours. Forty-eight hours later, the transgene (GFP) expression was quantified by flow cytometry. Data presented in figure is a mean of 3 replicates. Error bars represent SD, n−3, ***P≤0.001.

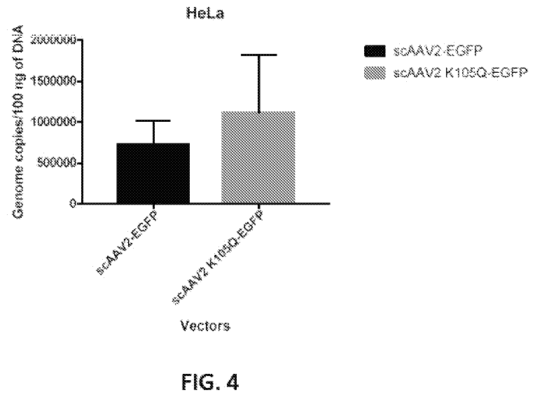

In one embodiment of the present invention, further assessed the entry profile of scAAV2 K105Q and found that entry of K015Q mutant was similar to WT-AAV2 (FIG. 4). Taken together, these data suggest that the increased transduction seen with K105Q mutant may be due to decreased SUMOylation of AAV2 capsid during its packaging or viral trafficking.

FIG. 4 is a graph depicting entry profile of AAV2 wild-type and K105Q mutant vector in HeLa cells in vitro, in the context of the present invention. HeLa cells were seeded in 4 replicates at density of 1×10⁵ cells per well in a 24 well plate. Cells were mock infected or infected with scAAV2-EGFP and scAAV2 K105Q-scEGFP mutant vector at an MOI of 1×10⁴ vgs/cell. Cells were collected 3 hours post-infection and genomic DNA was isolated as described in the methods. Viral genomes were titered against the plasmid reference standard. Data presented in the figure is a mean of 3 biological replicates with 3 technical replicates for each condition. Error bars represent SD, n=9.

### AAV2 Mutant Capsid Modified at a SUMOylation Site Demonstrates Reduced Levels of SUMO-1 Protein.

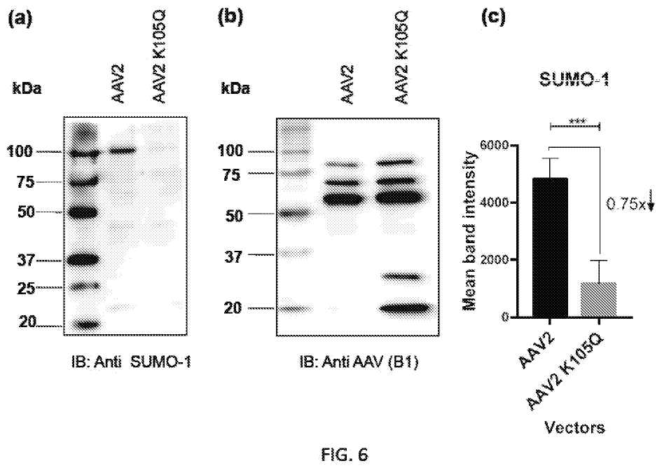

Since the SUMOylation site modified (K105Q) AAV2 vector, had a consistent increase in transduction in multiple cell lines, to determine the mechanistic basis of this phenotype studies were performed. The levels of SUMO-1 protein was assessed in the freshly packaged AAV2-K105Q and AAV2 wildtype vectors by immunoblotting (FIG. 6). Our data shows that the AAV2 K105Q vector had a significantly lower amount (˜75%) of SUMOylation in comparison to AAV2-wildtype vectors. This observation also confirms that K105 site is a major target of SUMOylation in AAV2 capsid.

### SUMOylation Site Modified Vector Enhances Factor-IX Expression in Hemophilia B Mice

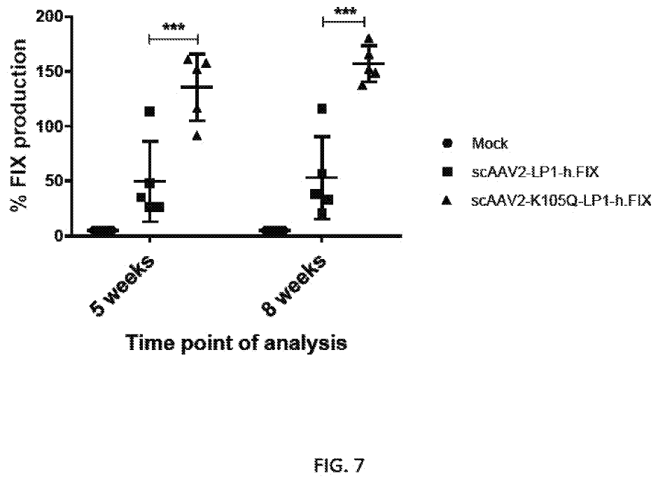

In one embodiment of the present invention, after screening for the efficiency of mutant vectors in vitro, the hepatic gene delivery potential of K105Q vector in an in vivo model is determined. We thus mock-injected or injected AAV2 wild type and K105Q mutant vectors expressing FIX into hemophilia B mice at the dose of 5×10¹⁰ vgs per animal (n=5). After 5 weeks of vector administration, F-IX levels in plasma were determined by ELISA. The media F-IX levels in animals that received the mutant K105Q was 135.8%±30.37%, whereas in animals that received the wild type vectors had F-IX levels of 49.83%±36.66%. (FIG. 7). These findings suggest the significant potential of SUMOylation mutant AAV2 vectors for hepatic gene therapy of hemophilia B. The FIG. 7 is graph plot depicting Factor-IX gene transfer in a murine model of hemophilia B. About 5×10¹⁰ vector genomes of scAAV2 LP1 F-IX and scAAV2 K105Q LP1 F-IX vectors were injected in Factor-IX deficient mice through intravenous route. After 5 weeks, plasma was collected after retro-orbital bleeding. An ELISA was performed for measuring factor-IX levels. Data presented in the FIG. 7 is a mean of 5 replicates. Error bars represent SD, n=5, ***P≤0.001.

### Neddylation and SUMOylation Mutant Vectors are not Immunogenic in Comparison to Wildtype AAV2 Vectors

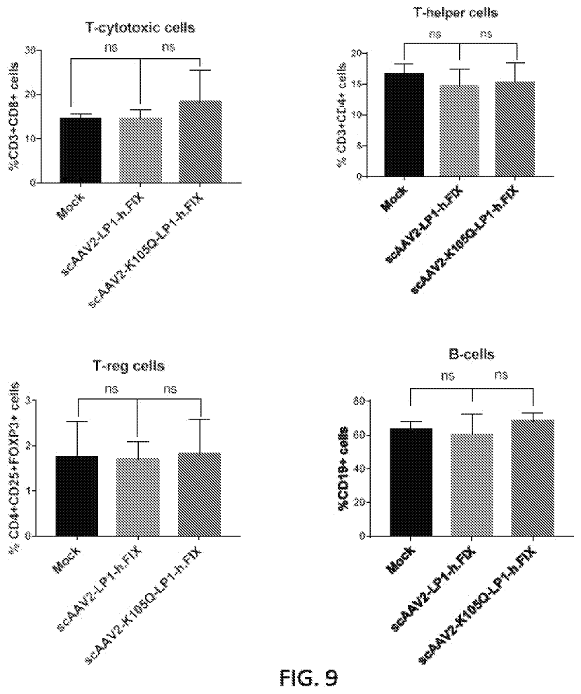

The next set of studies characterized the immune profile of the mutant AAV2-h.FIX vectors 9 weeks after hepatic gene transfer in hemophilia B mice. As can be seen in FIG. 9 a significant increase was not observed in the subpopulation of T cells including T-helper cells (14.67%±2.75 (AAV2) vs 15.32%±3.08 (K105Q), 14.05%±2.91 (K665Q); cytotoxic T cells (14.59%±2.07 (AAV2) vs 18.3%±7.22 (K105Q), 14.68%+3.62 (K665Q); or regulatory T cells (1.70%±0.38 (AAV2) vs 1.82%±0.76 (K105Q), 1.35%±0.34 (K665Q) between the mutant and wildtype AAV2 vector administered hemophilia B animals. A similar data was obtained when the B-cells were enumerated (FIG. 9).

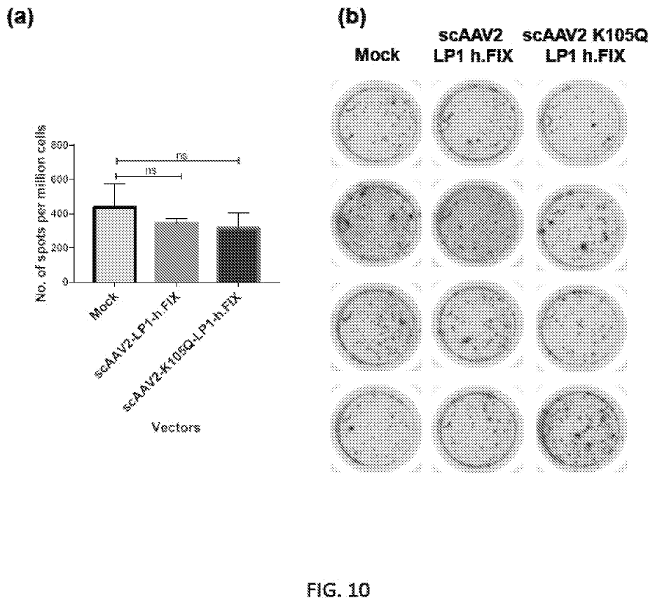

Furthermore, splenocytes were harvested from the mock treated and AAV2 treated mice and the capsid specific CD8+ T cell-based response was evaluated by the IFN-γ ELISPOT assay. The data shown in FIG. 10, demonstrates that Concanavalin A (positive control) generated ˜1100 number of spots per million stimulated splenocytes. Among the test groups, the IFN-γ response from splenocytes in animals that had gene transfer was at basal levels and was not significantly different between splenocytes of mice that received wildtype AAV2 or mutant AAV2 vectors. This data suggests that in murine models of hemophilia B, the host T-cell response against AAV2 vectors is negligible after a single, low dose of AAV2 vectors.

### Mutant AAV2 Vector Improves Efficiency of Ocular Gene Transfer in Mouse Retina

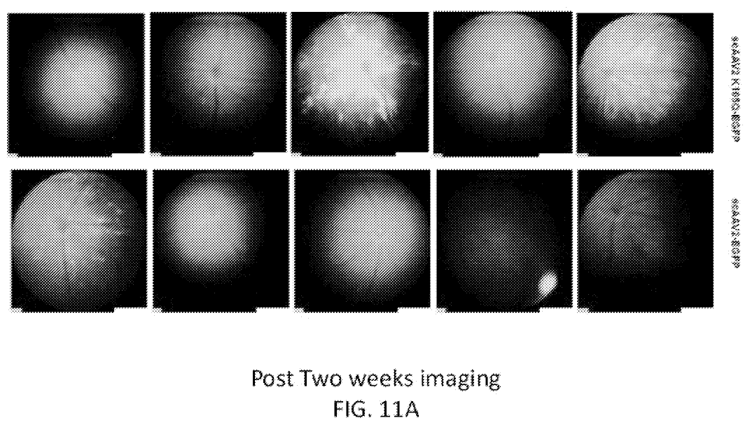

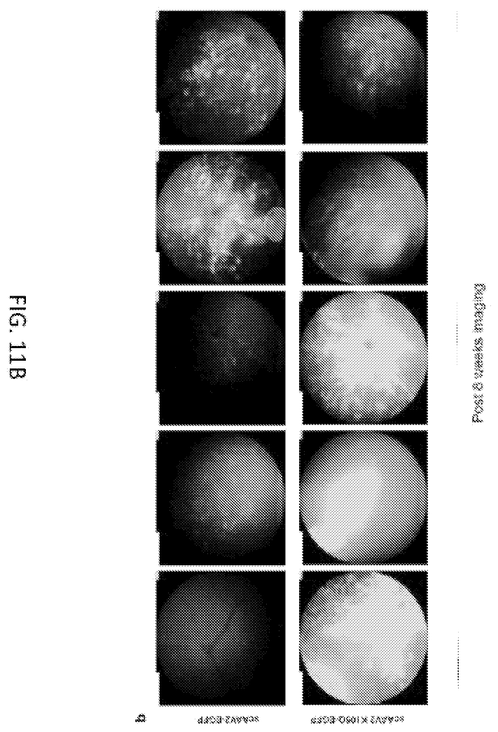

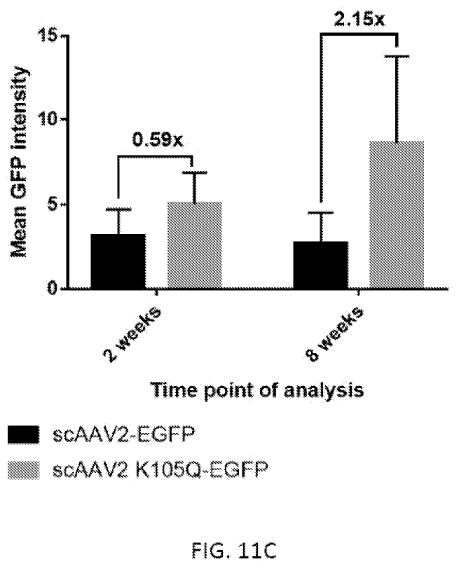

In another embodiment, to study the potential of SUMOylation mutant in another tissue type, the AAV2 wild type and mutant vector were administered, via the intravitreal route into normal the eyes of normal C57BL6/J mice. scAAV2-WT or the K105Q mutant vector containing EGFP transgene was injected in 1 μl volume at a dose of 3×10⁸ vgs/eye (n=6 eyes per group). Consistent with our in vitro data, the K105Q mutant had 0.59 to 2.15-fold higher EGFP expression levels (FIG. 6A-C), over a period of two to eight weeks. FIGS. 11A-B are images of Ocular gene transfer in C57BL6/J mice with SUMOylation mutant vector, respectively for 2 and 8 weeks. FIG. 11C is graph plot between mean GFP intensity and time plot analysis for Ocular gene transfer in C57BL6/J mice with SUMOylation mutant vector, respectively for 2 and 8 weeks. Eyes of C57BL6/J mice were mock injected or injected with scAAV2-EGFP and scAAV2 K105Q-EGFP by intravitreal route. For imaging, eyes were dilated by 1% Atropine, Phenylephrine+Tropicamide. Mice were anesthetized by intraperitoneal injection of ketamine (80 mg/kg) and xylazine (12 mg/kg) and 2.5% Hypromellose was applied to the eye before imaging. Intensity was set at maximum and gain was set at 15 db, frame rate was set at 6 fps for imaging of all the groups. Imaging was done at 2 and 8 weeks after injection. Image analysis was done by using Concentric Circle Plugin in ImageJ software. n=5 eyes.

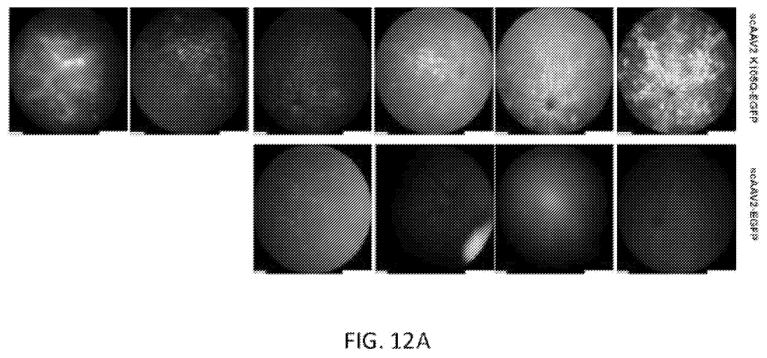

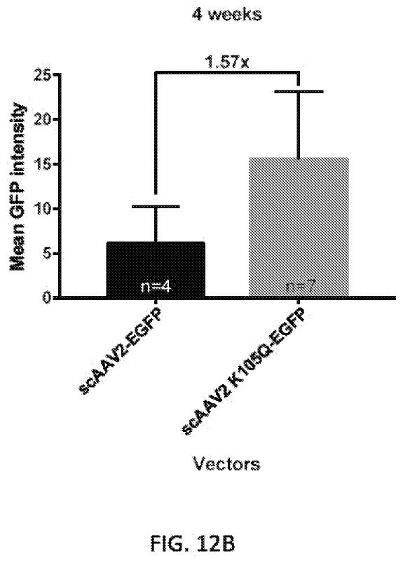

To further check if this increase in consistent with multiple vector packaging, all the viruses were repackaged and performed a second set of intra-ocular administrations with the same vector dose into C57BL6/J mice (n=8 eyes per group). Four weeks after intra-ocular gene transfer, it was observed a 1.57-fold increase in EGFP expression in AAV2-K105Q mutant administered mice in comparison to AAV2 wild type vector administered mice (FIG. 12A-B). It should be noted that some of the AAV2 and AAV2 K105Q vector injected eyes, did not show any EGFP expression which is possibly due to variations in the manual injection procedure. The present data underscores the possible utility of AAV2 K105Q vector for ocular gene transfer applications. FIG. 12A are images depicting Ocular gene transfer in C57BL6/J mice with SUMOylation mutant vector four weeks after gene transfer. FIG. 12B is graph plot illustrating Ocular gene transfer in C57BL6/J mice with SUMOylation mutant vector four weeks after gene transfer. Eyes of C57BL6/J mice were mock injected or injected with scAAV2-EGFP and scAAV2 K105Q-EGFP by intravitreal route. For imaging, eyes were dilated by 1% Atropine, Phenylephrine+Tropicamide. Mice were anesthetized by intraperitoneal injection of ketamine (80 mg/kg) and xylazine (12 mg/kg) and 2.5% Hypromellose was applied to the eye before imaging. Intensity was set at maximum and gain was set at 18 db, Frame rate was set at 4 fps for imaging of all the groups. Imaging was done at post 4 week. Image analysis was done by using Concentric Circle Plugin in ImageJ software. n=4 eyes for scAAV2-EGFP and n=7 for scAAV2 K105Q-EGFP.

AAV2-K105Q Vectors Encoding RPE65 Demonstrate Phenotypic Correction after Subretinal Gene Transfer in Rd12 Mice

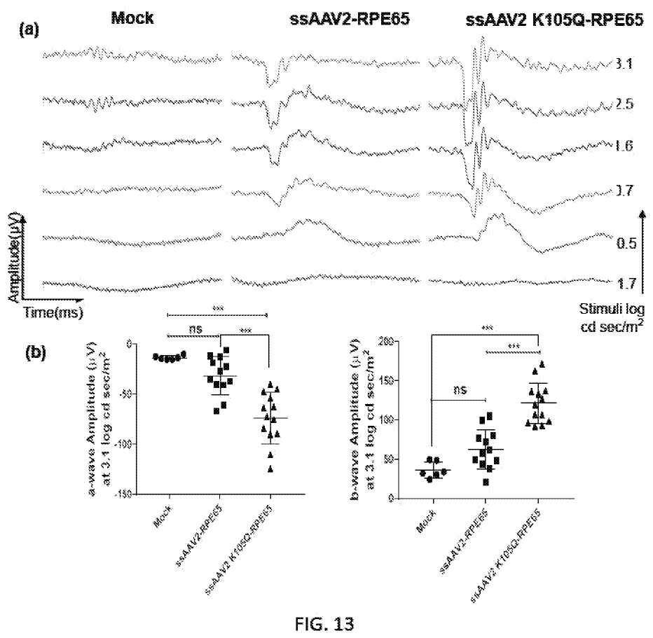

To further evaluate the therapeutic efficiency of AAV2-K105Q vectors for ocular gene therapy, AAV2 wild type and K105Q expressing human retinal pigmental epithelium gene encoding 65 kDa protein (RPE65) was administered in groups of rd12 mice. Approximately, 1-2 μl of vectors containing 7×10⁸ vgs were administered by subretinal route into the eyes of rd12 mice. The phenotypic rescue was measured by electroretinography (ERG) analysis 10 weeks after vector administration. Representative ERG waveforms are shown in FIG. 13. The A-wave amplitude for K105Q vector administered mice was significantly elevated to −63±9.3 μV in comparison to eyes that received AAV2 wild type vectors (−25.07±12.69 μV) and mock injected animals (−13.4±2.0 μV) (FIG. 13b). The B-wave amplitude for AAV2 K105Q administered and AAV2 wild type administered eyes were 93.69±2.0 μV and 55.07±15.12 μV, respectively (FIG. 13b). This highlights that ocular gene therapy with AAV2 K105Q-RPE65 vectors has a therapeutic A-wave amplitude response and B-wave response when compared to rd12 mice that received wild type AAV2 vectors.

AAV are becoming an indispensable vector in a field of gene therapy, particularly when targeting to post-mitotic tissues such as the liver or eye is involved. Nonetheless, since immune and transduction barriers remain. The present invention has employed multiple strategies to overcome them. The goal of each of the known strategies is to target the underlying molecular mechanisms responsible for inhibition of AAV transduction or intracellular events that lead to degradation of capsid proteins and its clearance by immune effector cells. One of the active molecular mechanism which involves the capsid degradation and may determine the fate of AAV transduction is posttranslational modifications (PTMs) of AAV capsid proteins.

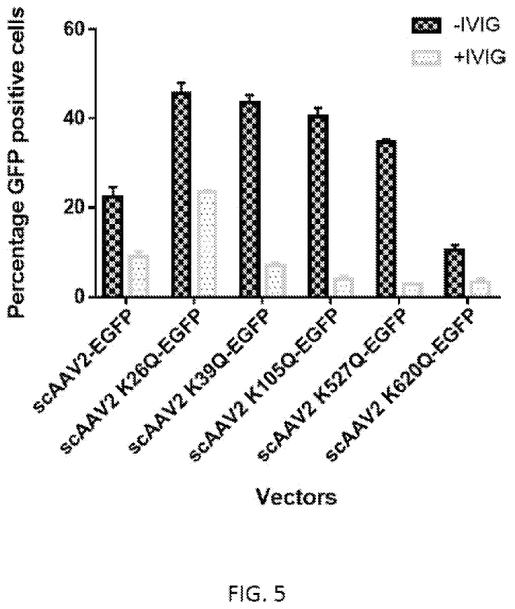

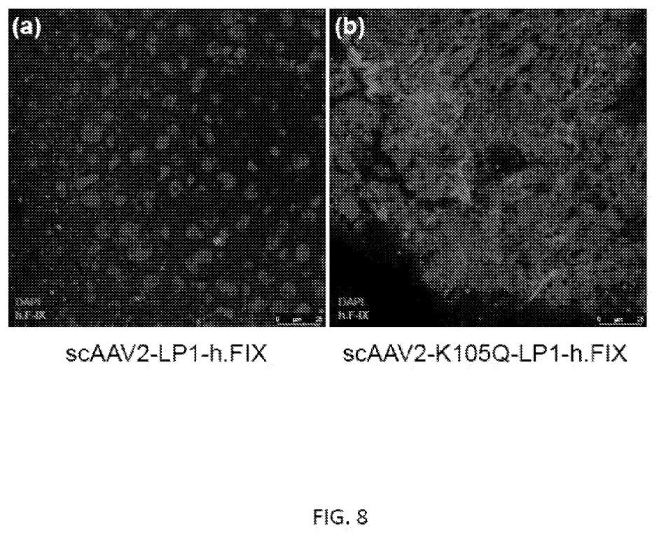

Considering the comprehensive role of SUMOylation in numerous biological process as discussed above and based our secondary analysis of a publicly available dataset which highlights the up regulation of most of the genes involved in SUMOylation pathway upon AAV2 transduction (Table-1), the present invention provides for novel AAV vectors by modifying the capsid amino acids that are potential substrates for SUMOylation. The present invention used in silico prediction tools, i.e. GPS-SUMO and SUMOplot to predict the possible sites for SUMOylation on AAV2 VP1 capsid protein sequence. The present invention has chosen the consensus sites predicted between GPS-SUMO and SUMOplot and sites having a score >0.5, to achieve a high degree of confidence from the prediction analysis. The five short-listed lysinc residues (K26, K39, K105, K527, K620) were mutated to glutamine, and among these vectors, K105Q mutant showed significant increase (44% to 110%) in multiple cell lines (HeLa, Huh7 and ARPE-19) in vitro. The present invention also provides for evaluation of the mechanisms by which AAV2-K105Q mutant had a high transduction efficiency, for this viral entry assay was performed to study its internalization pattern in HeLa cells. The entry profile of K105Q mutant was similar to WT-AAV2 vectors (FIG. 4), thus suggesting the role of intracellular events such as SUMOylation during AAV trafficking contributing to its increase in transduction. However, further detailed studies are needed to ascertain the basis of this improved transduction seen with AAV2-K105Q mutant vectors. To further evaluate the immune escape function of all the five mutant vectors, an in vitro neutralization assay was performed (FIG. 8). During evaluation any significant neutralization antibody escape potential of present mutant AAV2 vectors was not observed, further in vivo studies are warranted to understand their impact on T-cell immune response, since SUMOylation is a major trigger to activation of T-cell response. FIG. 5 is graph plot showing a virus neutralization assay. Approximately 3×10⁴ Hela cells were seeded per well in triplicates and incubated for 12 hours in Iscove's-modified Dulbecco's medium with 10% FBS. All vectors were incubated with 1:256 dilution of IVIG (10 mg/ml) for 1 hour at 37° C. Cells were mock-infected or infected with mutants and scAAV2-EGFP along with their IVIG treated groups at an MOI of 5×10³ vgs/cell. Forty-eight hours later, the transgene (GFP) expression was quantified by flow cytometry. Representative data selected from 4 independent experiments is shown. Error bars represent SD, n=3 replicates.

The present also performed hepatic and ocular gene transfer expressing human coagulation F-IX or EGFP, respectively, to further understand if AAV2 K105 Q vectors have therapeutic potential in vivo. AAV2-K105Q mutant demonstrated a significant increase in F-IX levels in hemophilia B mice with the median F-IX level of 152.3% (range: 91.77%-160.9%) as compared to WT-AAV2 [median−35.19% (26.04%-113.4%)] injected hemophilia B mice. For the intraocular testing, scAAV2-K105Q-EGFP vectors showed a significant increase in transduction by 0.59 to 2.15-fold in mouse retina when analysed by fundus imaging in a Micron IV imaging system (FIGS. 12A-B and FIGS. 11A-C). Taken together, the identification of an AAV2 mutant that demonstrates improved phenotypic expression in vivo in two of the most studied organs (ocular or hepatic) for gene transfer is likely to improve the repertoire of AAV2 vectors available for gene therapy.

The present invention unravels the hitherto unknown roles of SUMOylation specific amino acids on AAV2 capsid protein. Furthermore, the development of SUMOylation target modified AAV2 vectors presents an exciting opportunity for hepatic or ocular gene transfer with the safest AAV vector in human gene therapy applications.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Also, those acts that are not dependent on other acts may be performed in parallel with the other acts. The scope of embodiments is by no means limited by these specific examples.

1. A process for producing a plurality of SUMOylation target-site modified AAV vectors, comprising:
predicting a plurality of SUMOylation target-sites on AAV VP1 capsid protein sequence based on direct amino acid match to SUMO-consensus sequence and substitution of the consensus amino acid residues with amino acid residues exhibiting similar hydrophobicity,
wherein the AAV VP1 capsid protein sequence being set forth in Protein ID-YP 680426.1;
scoring each of the plurality of SUMOylation target-sites on AAV VP1 capsid protein sequence;
producing the plurality of SUMOylation target-site modified AAV vectors based on predicted plurality of SUMOylation target-sites on AAV VP1 capsid protein sequence,
wherein the producing of the plurality of SUMOylation target-site modified AAV vectors comprises selecting the predicted plurality of SUMOylation target-sites on AAV VP1 capsid protein sequence, having score >0.5, for site-directed mutagenesis, and mutating the selected plurality of SUMOylation target-sites from Lysine (K) to Glutamine (Q) residues to produce plurality of SUMOylation target-site modified AAV vectors.

2. The process as claimed in claim 1, wherein the AAV is selected from a group consisting AAV serotypes 1, 2, 3, 4, 5, 6, 7, 8, 9, rhlO and other AAV variants thereof.

3. The process as claimed in claim 1, wherein the AAV is AAV2.

4. The process as claimed in claim 1, wherein the plurality of SUMOylation target-sites on AAV2 VP1 capsid protein sequence are predicted in-silico.

5. The process as claimed in claim 1, wherein the selected plurality of SUMOylation target-sites subject to mutation comprises lysine residues at K26, K39, K105, K527, K620 in the VP1 protein.

6. The process as claimed in claim 1, wherein mutating the selected plurality of SUMOylation target-sites comprises mutating lysine residues at the K26, K39, K105, K527, K620 in the VP1 protein to corresponding glutamine residues.

7. The process as claimed in claim 1, wherein mutating the selected plurality of SUMOylation target-sites is performed using a set of primers designed for mutating the selected sites from Lysine (K) to Glutamine (Q) or other similar aminoacids, where the set of primers has been set forth in SEQ ID NOs. 1 to 10

8. The process as claimed in claim 1, wherein the plurality of SUMOylation target-site modified AAV2 vectors comprises K26Q, K39Q, K105Q, K527Q, K620Q containing transgenes.

9. The process as claimed in claim 1, wherein the plurality of SUMOylation target-site modified AAV vectors possess significantly higher gene expression, with respect to wild type, thereby improving efficiency of hepatic and ocular gene transfer.

10. A plurality of SUMOylation target-site modified AAV vectors produced by the process of claim 1.

11. The plurality of SUMOylation target-site modified AAV vectors as claimed in claim 10, wherein the plurality of SUMOylation target-site modified AAV vectors are not immunogenic in comparison to wildtype AAV2 vectors.
